(19) United States
(12) Patent Application Publication
(10) Pub. No.: US 2006/0036331 A1
(43) Pub. Date: Feb. 16, 2006

(54) Polymer-ceramic-hydrogel composite scaffold for osteochondral repair

(76) Inventors: Helen H. Lu, New York, NY (US); Jie Jiang, New York, NY (US); Clark T. Hung, Ardsley, NY (US); X. Edward Guo, New York, NY (US); Gerard Ateshian, New York, NY (US)

Correspondence Address: COOPER & DUNHAM, LLP, 1185 AVENUE OF THE AMERICAS, NEW YORK, NY, 10036, US

(21) Appl. No.: 11/073,261

(22) Filed: Mar. 4, 2005

Related U.S. Application Data
(60) Provisional application No. 60/550,809, filed on Mar. 5, 2004.

(51) Int. Cl. A61F 2/28; C12N 5/08

(52) U.S. Cl. 623/23.51; 435/397; 623/23.76; 623/23.63

(57) ABSTRACT

This invention pertains to materials and methods relating to the biological fixation of one tissue type to another different tissue type, i.e., the fixation of cartilage to bone. A scaffold apparatus for osteochondral tissue engineering is described. The apparatus comprises regions of varying matrices which provide a functional interface between multiple tissue types. Further, a method for preparing the scaffold apparatus is provided. Methods for treating osteochondral tissue injury and cartilage degeneration using the scaffold apparatus are also described. In addition, a method for evaluating cell-mediated and scaffold-related parameters of development and maintenance of multiple tissue zones in vitro is described.

This application claims the benefit of U.S. Provisional Application No. 60/550,809, filed Mar. 5, 2004, the entire contents of which are incorporated herein by reference.

Throughout this application, various publications are referred to by arabic numerals within parentheses. Full citations for these publications are presented in a References section immediately before the claims. Disclosures of the publications cited in the References section in their entireties are hereby incorporated by reference into this application in order to more fully describe the state of the art as of the date of the methods and apparatuses described herein.

### BACKGROUND OF THE INVENTION

This application relates to osteochondral repair. For example, a scaffold apparatus is discussed below which can serve as a functional interface between cartilage and bone. Methods for preparing a multi-region scaffold are also discussed.

As an example of cartilage-bone interface, the human osteochondral interface is discussed below to aid in understanding the discussion of the methods and apparatuses of this application.

Arthritis is a condition caused by cartilage degeneration that affects many adults, and it is the primary cause of disability in the United States. Clinical intervention is typically required, since cartilage injuries generally do not heal.

Osteoarthritis involves pathological mineralization of articular cartilage which causes cartilage surface depletion. Articular cartilage has an instrinsically poor repair potential, and clinical intervention is often required. Cartilage injuries to the subchondral bone typically undergo partial repair. Some repair techniques include cell-based therapy, subchondral drilling and total joint replacement. However, such current techniques do not fully restore the functionality of the osteochondral interface.

Osteochondral grafting is another repair technique. Tissue engineered osteochondral grafts have been disclosed (Sherwood et al. 2002; Gao et al. 2001, 2002; Schafer et al. 2000, 2002). An osteochondral graft may improve healing while promoting integration with host tissue.

Calcium phosphates have been shown to modulate cell morphology, proliferation and differentiation. Calcium ions can serve as a substrate for Ca²⁺-binding proteins, and modulate the function of cytoskeleton proteins involved in cell shape maintenance.

Gregiore et al. (1987) examined human gingival fibroblasts and osteoblasts and reported that these cells underwent changes in morphology, cellular activity, and proliferation as a function of hydroxyapatite particle sizes. Culture distribution varied from a homogenous confluent monolayer to dense, asymmetric, and multi-layers as particle size varied from less than 5 μm to greater than 50 μm, and proliferation changes correlated with hydroxyapatite particles size.

Cheung et al. (1985) further observed that fibroblast mitosis is stimulated with various types of calcium-containing complexes in a concentration-dependent fashion.

Chondrocytes are also dependent on both calcium and phosphates for their function and matrix mineralization. Wuthier et al. (1993) reported that matrix vesicles in fibrocartilage consist of calcium-acidic phospholipids-phosphate complex, which are formed from actively acquired calcium ions and an elevated cytosolic phosphate concentration.

Phosphate ions have been reported to enhance matrix mineralization without regulation of protein production or cell proliferation, likely because phosphate concentration is often the limiting step in mineralization. It has been demonstrated that human foreskin fibroblasts when grown in micromass cultures and under the stimulation of lactic acid can dedifferentiate into chondrocytes and produce type II collagen.

Scaffold devices for insertion of implants in the cartilage bone interface have been proposed. See, for example, U.S. patent application No. US 2003/0114936A1 and U.S. Pat. No. 6,454,811.

However, there is a need for an improved scaffold apparatus which can be used in an in vitro graft system for regenerating the osteochondral interface.

### SUMMARY

This disclosure provides an apparatus for osteochondral tissue engineering, wherein said apparatus comprises regions of varying matrices which provide a functional interface between multiple tissue types, said regions comprising, according to one embodiment, (a) a first regions comprising a hydrogel, (b) a second region adjoining the first regions, and (c) a third region adjoining the second region and comprising a porous scaffold.

This disclosure also comprises a method for treating osteochondral tissue injury in a subject comprising, according to one embodiment, grafting an apparatus with a co-culture of two or more cells selected from the group comprising chondrocytes, osteoblasts, osteoblast-like cells and stem cells in the subject at the location of osteochondral tissue injury.

This disclosure also comprises a method for treating cartilage degeneration in a subject comprising, according to one embodiment, grafting an apparatus with a co-culture of two or more cells selected from the group comprising chondrocytes, osteoblasts, osteoblast-like cells and stem cells in the subject at the location of cartilage degeneration.

This disclosure further comprises a method, according to one embodiment, for evaluating cell-mediated and scaffold-related parameters for development and maintenance of multiple tissue zones in vitro comprising (a) co-culturing cells of different tissue on an apparatus and (b) after a suitable period of time, examining the development and maintenance of the cells on the apparatus.

In addition, this disclosure provides a method for preparing an apparatus for osteochondral tissue engineering, said method comprising the steps of (a) using a mold to form an apparatus comprising a first region comprising hydrogel, a second region adjoining said first region, and a third region adjoining said second region and comprising a porous scaffold, (b) seeding said first region with one or more cells for chondrogenesis, (c) seeding said third region with one or more cells for osteogenesis and (d) maintaining the apparatus comprising the first region seeded with the cells for chondrogenesis and the third region seeded with the cells for osteogenesis in an environment supporting migration of at least some of the cells for chondrogenesis into the second region and migration of at least some of the cells for osteogenesis into the second region.

### BRIEF DESCRIPTION OF THE FIGURES

FIG. 1

A block diagram of an apparatus for osteochondral tissue engineering, according to one embodiment.

FIG. 2

A flow chart for a method for preparing an apparatus for osteochondral tissue engineering, according to one embodiment.

FIG. 3

Osteochondral Composite (G=Gel, I=Interface, M=Microsphere)

FIG. 4

(A) Bovine chondrocyte growth on 25% PLAGE-BG composite scaffolds. (B) Effects of BG content on alkaline phosphatase (ALP) activity of chondrocytes.

FIG. 5

Matrix organization on the osteochondral construct after 10 days of culture. (A) GAG deposition (blue). (B) Collagen (red). (C) Mineralization (red). (Co=Collagen, CH=Chondrocyte, M=Microsphere, G=Gel, 20×)

FIG. 6

(Left) Micro-CT scan of the osteochondral construct, and (Right) EDAX spectrum of the Interface region indicate that mineralization was limited to the Interface (I) and Microsphere (M) regions.

FIG. 7

Preparation of sample using a water-oil-water emulsion method.

FIG. 8

Effects of BG % on chondrocyte growth.

FIG. 9

Media pH measurements for 25% BG composites.

FIG. 10

ALP activity for 25% BG composites and 0% BG composites.

FIG. 11

GAG content for 25% BG composites and 0% BG composites.

FIG. 12

Histological stains of day 28 scaffolds (A) Trichrome of PLAGA-BG (10×), (B) Von Kossa of PLAGA-BG (10×).

FIG. 13

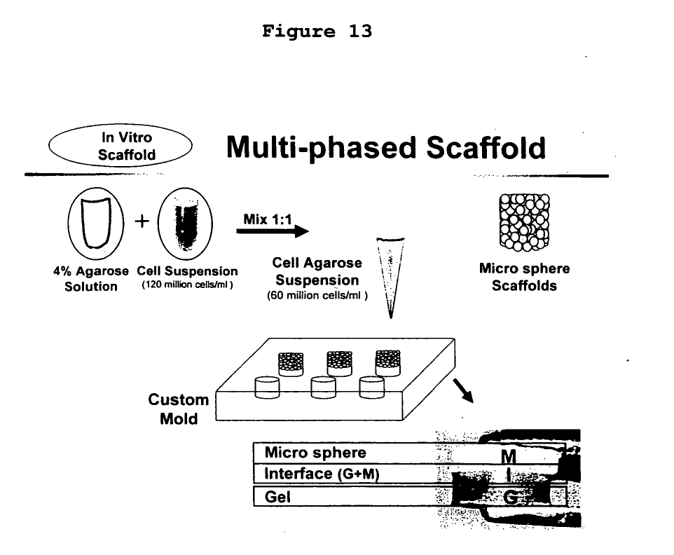

Diagram illustrating one embodiment for preparing a multiphased apparatus.

### DETAILED DESCRIPTION OF THE INVENTION

Definitions

In order to facilitate an understanding of the material which follows, one may refer to Freshney, R. Ian. Culture of Animal Cells—A Manual of Basic Technique (New York: Wiley-Liss, 2000) for certain frequently occurring methodologies and/or terms which are described therein.

However, except as otherwise expressly provided herein, each of the following terms, as used in this application, shall have the meaning set forth below.

As used herein, “bioactive” shall include a quality of a material such that the material has an osteointegrative potential, or in other words the ability to bond with bone. Generally, materials that are bioactive develop an adherent interface with tissues that resist substantial mechanical forces.

As used herein, “biomimetic” shall mean a resemblance of a synthesized material to a substance that occurs naturally in a human body and which is not rejected by (e.g., does not cause an adverse reaction in) the human body.

As used herein, “chondrocyte” shall mean a differentiated cell responsible for secretion of extracellular matrix of cartilage.

As used herein, “chondrogenesis” shall mean the formation of cartilage tissue.

As used herein, “fibroblast” shall mean a cell of connective tissue, mesodermally derived, that secretes proteins and molecular collagen including fibrillar procollagen, fibronectin and collagenase, from which an extracellular fibrillar matrix of connective tissue may be formed.

As used herein, “hydrogel” shall mean any colloid in which the particles are in the external or dispersion phase and water is in the internal or dispersed phase. For example, a chondrocyte-embedded agarose hydrogel may be used in some instances. As another example, the hydrogel may be formed from hyaluronic acid, chitosan, alginate, collagen, glycosaminoglycan and polyethylene glycol (degradable and non-degradable), which can be modified to be light-sensitive. It should be appreciated, however, that other biomimetic hydrogels may be used instead.

As used herein, “matrix” shall mean a three-dimensional structure fabricated from biomaterials. The biomaterials can be biologically derived or synthetic.

As used herein, “osteoblast” shall mean a bone-forming cell that is derived from mesenchymal osteoprognitor cells and forms an osseous matrix in which it becomes enclosed as an osteocyte. The term is also used broadly to encompass osteoblast-like, and related, cells, such as osteocytes and osteoclasts.

As used herein, “osteogenesis” shall mean the production of bone tissue.

As used herein, “osteointegrative” shall mean having the ability to chemically bond to bone.

As used herein, “polymer” shall mean a chemical compound or mixture of compounds formed by polymerization and including repeating structural units. Polymers may be constructed in multiple forms and compositions or combinations of compositions.

As used herein, “porous” shall mean having an interconnected pore network.

As used herein, “subject” shall mean any organism including, without limitation, a mammal such as a mouse, a rat, a dog, a guinea pig, a ferret, a rabbit and a primate. In the preferred embodiment, the subject is a human being.

As used herein, “treating” a subject afflicted with a disorder shall mean causing the subject to experience a reduction, remission or regression of the disorder and/or its symptoms. In one embodiment, recurrence of the disorder and/or its symptoms is prevented. In the preferred embodiment, the subject is cured of the disorder and/or its symptoms.

### EMBODIMENTS OF THE INVENTION

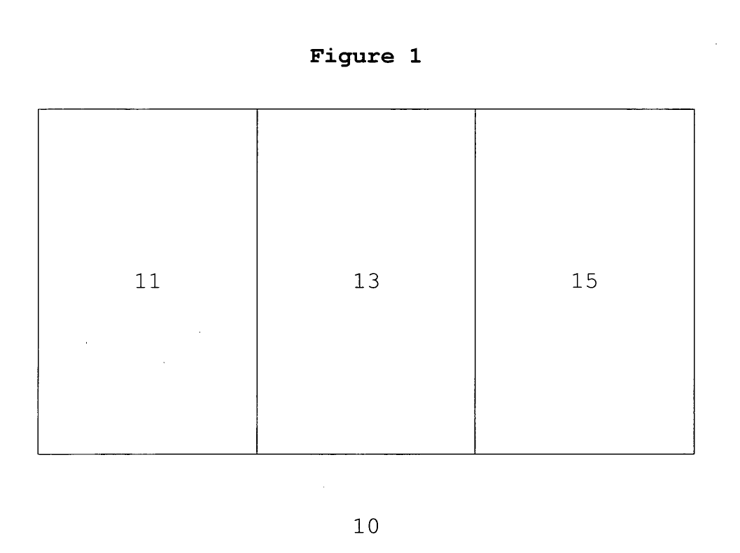

This disclosure provides an apparatus for osteochondral tissue engineering. According to one embodiment (FIG. 1), an apparatus 10 comprises regions 11, 13 and 15 of varying matrices which provide a functional interface between multiple tissue types. The first region 11 comprises a hydrogel. The second region 13 adjoins the first region 11. The third region 15 adjoins the second region 13 and comprises a porous scaffold.

The apparatus preferably promotes the growth and development of multiple tissue types. In one exemplary embodiment, the first region 11 is seeded with cells for chondrogenesis, the third region 15 is seeded with cells for osteogenesis, and the apparatus 10 comprising the first region 11 seeded with the cells for chondrogenesis, and the third region 15 seeded with the cells for osteogenesis is maintained in an environment supporting migration of at least some of the cells for chondrogenesis into the second region 13 and migration of at least some of the cells for osteogenesis into the second region 13. The cells for chondrogenesis may include chondrocytes and/or stem cells. The chondrocytes can be selected from the group comprising surface zone chondrocytes, middle zone chondrocytes and deep zone chondrocytes. The cells for osteogenesis can include osteoblasts, osteoblast-like cells and/or stem cells.

In one embodiment, the first region 11 supports the growth and maintenance of cartilage tissue, the third region 15 supports the growth and maintenance of bone tissue, and the second region 13 functions as an osteochondral interfacial zone. The first region 11 for supporting the growth and maintenance of cartilage tissue may be seeded with chondrocytes and/or stem cells. In another embodiment, region 11 is rich in glycosaminoglycan. In another embodiment, one or more agents selected from the group comprising the following are introduced in the first region: anti-infectives; hormones; analgesics; anti-inflammatory agents; growth factors; chemotherapeutic agents; anti-rejection agents; and RGD peptides. In one embodiment, the growth factor introduced into the first region is Transforming Growth Factor-beta (TGF-beta). In another embodiment, the hydrogel of the first region is agarose hydrogel.

In one embodiment, the second region 13 supports the growth and maintenance of fibrocartilage. The second region may include a combination of hydrogel and the porous scaffold. In another embodiment, the second region is rich in glycosaminoglycan and collagen. In another embodiment, one or more growth factors selected from the following are introduced into the second region: Transforming Growth Factor-beta (TGF-beta), parathyroid hormone and insulin-derived growth factors (IGF).

In one embodiment, the third region 15 for supporting the growth and maintenance of bone tissue is seeded with at least one of osteoblasts, osteoblast-like cells and stem cells. In another embodiment, the third region 15 includes a mineralized collagen matrix. In another embodiment, the third region 15 contains at least one of osteogenic agents, osteogenic materials, osteoinductive agents, osteoinductive materials, osteoconductive agents, osteoconductive materials, growth factors and chemical factors. In one embodiment, the growth factors are selected from the group comprising Transforming Growth Factor-beta (TGF-beta), bone morphogenetic proteins, vascular endothelial growth factor, platelet-derived growth factor and insulin-derived growth factors (IGF).

In another embodiment, the third region 15 comprises a composite of polymer and ceramic. In another embodiment, the ceramic is bioactive glass. In another embodiment, the ceramic is calcium phosphatase. In another embodiment, the third region contains approximately 25% bioactive glass by weight.

In one embodiment, a gradient of calcium phosphate concentrations appears across the first, second and third regions. In another embodiment, the gradient of calcium phosphate is related to the percent of bioactive glass in the third region. In another embodiment, the calcium phosphate is selected from the group comprising tricalcium phosphate, hydroxyapatite and a combination thereof.

In one embodiment, the polymer in the third region is selected from the group comprising aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, and biopolymers, and a blend of two or more of the preceding polymers. In another embodiment, the polymer comprises at least one of the poly(lactide-co-glycolide), poly(lactide) and poly(glycolide).

In one embodiment, the apparatus is biodegradable. In another embodiment, the apparatus is osteointegrative.

This disclosure also provides a method for treating osteochondral tissue injury in a subject. The method, according to one embodiment, includes grafting apparatus 10 with a co-culture of two or more cells selected from the group comprising chondrocytes, osteoblasts, osteoblast-like cells and stem cells in the subject at the location of osteochondral tissue injury. In one embodiment, the osteochondral tissue injury is craniofacial tissue injury. In another embodiment, the osteochondral injury is musculoskeletal tissue injury. In one embodiment, the chondrocytes are selected from the group comprising surface zone chondrocytes, middle zone chondrocytes and deep zone chondrocytes.

This disclosure also provides a method for treating cartilage degeneration in a subject. The method, according to one embodiment, includes grafting apparatus 10 with a co-culture of two or more cells selected from the group comprising chondrocytes, osteoblasts, osteoblast-like cells and stem cells in the subject at the location of cartilage degeneration. In one embodiment, the cartilage degeneration is caused by osteoarthritis. In one embodiment, the chondrocytes are selected from the group comprising surface zone chondrocytes, middle zone chondrocytes and deep zone chondrocytes.

This invention also provides a method for evaluating cell-mediated and scaffold-related parameters of development and maintenance of multiple tissue zones in vitro. The method, according to one embodiment, includes (a) co-culturing cells of different tissue on apparatus 10 and (b) after a suitable period of time, examining the development and maintenance of the cells on the apparatus. In one embodiment, the cells of different tissues comprise two or more of the cells selected from the group comprising chondrocytes, osteoblasts, osteoblast-like cells and stem cells. In one embodiment, the chondrocytes are selected from the group comprising surface zone chondrocytes, middle zone chondrocytes and deep zone chondrocytes. In another embodiment, the parameters of development and maintenance comprise cell proliferation, alkaline phosphatase activity, glycosaminoglycan deposition, mineralization, cell viability, scaffold integration, cell morphology, phenotypic expression, and collagen production.

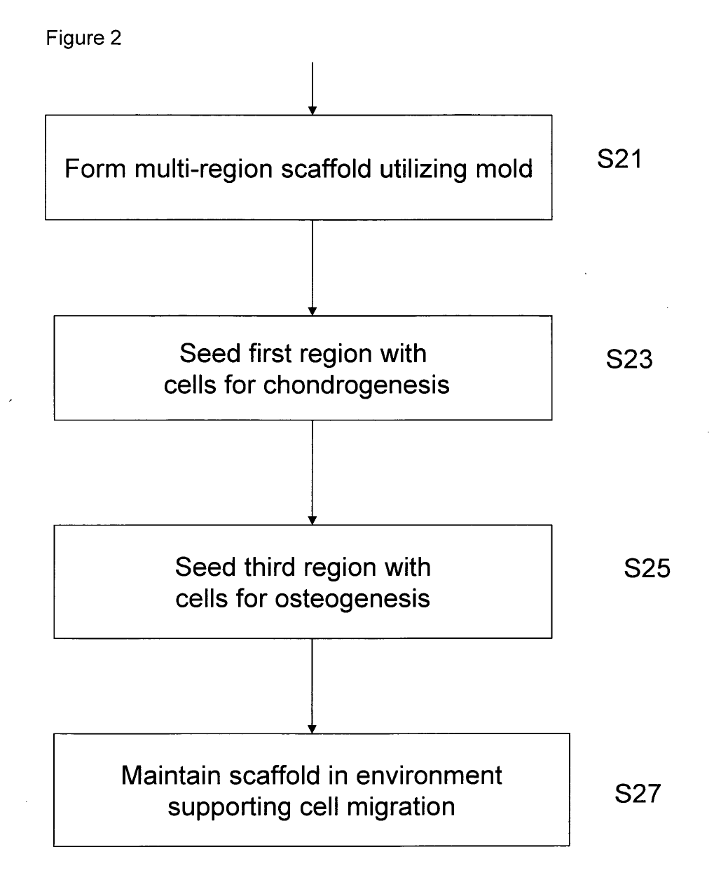

This disclosure also provides a method for preparing an apparatus for osteochondral tissue engineering. The method, according to one embodiment (FIG. 2), includes the steps of (a) using a mold to form an apparatus comprising a first region comprising hydrogel, a second region adjoining said first region, and a third region adjoining second region and comprising a porous scaffold (step S21), (b) seeding said first region with one or more cells for chondrogenesis (Step S223), (c) seeding said third region with one or more cells for osteogenesis (Step S25) and (d) maintaining the apparatus comprising the first region seeded with the cells for chondrogenesis and the third region seeded with the cells for osteogenesis in an environment supporting migration of at least some of the cells for chondrogenesis into the second region and migration of at least some of the cells for osteogenesis into the second region (Step S27).

The cells for chondrogenesis can include chondrocytes and/or stem cells. In one embodiment, the chondrocytes are selected from the group comprising surface zone chondrocytes, middle zone chondrocytes and deep zone chondrocytes. In another embodiment, the first region supports the growth and maintenance of cartilage tissue, the third region supports the growth and maintenance of bone tissue, and the second regions functions as an osteochondral interfacial zone. In another embodiment, the cells for osteogenesis include osteoblasts, osteoblast-like cells and/or stem cells.

In one embodiment of the method, the first region is rich in glycosaminoglycan. In another embodiment, the method further comprises the step of introducing in said first region one or more agents selected from a group comprising the following: anti-infectives; hormones; analgesics; anti-inflammatory agents; growth factors; chemotherapeutic agents; anti-rejection agents; and RGD peptides. In one embodiment, the growth factor introduced in to the first zone is Transforming Growth Factor-beta (TGF-beta). In another embodiment, the hydrogel of the first region is agarose hydrogel.

In one embodiment of the method, the second region supports the growth and maintenance of fibrocartilage. In another embodiment, the second region includes a combination of hydrogel and the porous scaffold. In another embodiment, the second region is rich in glycosaminoglycan and collagen. In another embodiment, one or more growth factors selected from the following are introduced into the second region: Transforming Growth Factor-beta (TGF-beta), parathyroid hormone and insulin-derived growth factors (IGF).

In another embodiment of the method, the third region includes a mineralized collagen matrix. In another embodiment, in the third region contains at least one of osteogenic agents, osteogenic materials, osteoinductive agents, osteoinductive materials, osteoconductive agents, osteoconductive materials, growth factors and chemical factors. In one embodiment, the growth factors are selected from the group comprising Transforming Growth Factor-beta (TGF-beta), bone morphogenetic proteins, vascular endothelial growth factor, platelet-derived growth factor and insulin-derived growth factors (IGF).

In another embodiment, the third region comprises a composite of polymer and ceramic. In one embodiment, the ceramic is bioactive glass. In another embodiment, the ceramic is calcium phosphatase. In another embodiment, the third region includes approximately 25% bioactive glass by weight.

In another embodiment of the method, a gradient of calcium phosphate concentrations appear across said first, second and third regions. In one embodiment, the gradient of calcium phosphate concentrations is related to the percent of bioactive glass in the third region. In another embodiment, the calcium phosphate is selected from the group comprising tricalcium phosphate, hydroxyapatite, and a combination thereof.

In one embodiment, the polymer in the third region is selected from the group comprising aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, and biopolymers, and a blend of two or more of the preceding polymers. In another embodiment, the polymer comprises at least one of poly(lactide-co-glycolide), poly(lactide) and poly(glycolide).

In one embodiment of the method, the apparatus prepared though said method is biodegradable. In another embodiment, the apparatus prepared through said method is osteoinductive.

The specific embodiments described herein are illustrative, and many variations can be introduced on these embodiments without departing from the spirit of the disclosure or from the scope of the appended claims. For example, elements and/or features of illustrative embodiments may be combined with, and/or substituted for, each other within the scope of this disclosure and appended claims.

Further non-limiting details are described in the following Experimental Details section which is set forth to aid in an understanding of the invention but is not intended to, and should not be construed to, limit in any way the claims which follow thereafter.

Experimental Details

First Set of Experiments

In the past decade, tissue engineering has emerged as an alternative approach to implant design and tissue regeneration. Design methodologies adapted from current tissue engineering efforts can be applied to regenerate the osteochondral interface.

An in vitro graft system was developed for the regeneration of the osteochondral interface. The native osteochondral interface spans from nonmineralized cartilage to bone, thus one of the biomimetic design parameters for the multiphased osteochondral graft is the calcium phosphate (CA-P) content of the scaffold. The components of this graft system include (1) a hybrid scaffold of hydrogel and polymer-ceramic composite (PLAGA-BG), (2) novel co-culture of osteoblasts and chondrocytes, and (3) a multi-phased scaffold design comprised of three regions intended for the formation of three distinct tissue types: cartilage, interface, and bone. In the current design, the Ca-P content is related to the percent of BG in the PLAGA-BG composite. From the material selection standpoint, one phase of the hydrogel-polymer ceramic scaffold is based on a thermal setting hydrogel which has been shown to develop a functional cartilage-like matrix in vitro [3]. The second phase of the scaffold consists of a composite of polylactide-co-glycolide (PLAGA) and 45S5 bioactive glass (BG). PLAGA-BG is biodegradable, osteointegrative, and able to support osteoblast growth and phenotypic expression [2]. The middle phase, which interfaces the first and second, has a lower Ca—P content than the second phase, being of a mixture of the hydrogel and the PLAGA-BG composite.

The scaffolds utilized in this set of experiments are composed of PLAGA-BG microspheres fabricated using the methods of Lu et al. [2]. Briefly, PLAGA 85:15 granules were dissolved in methylene chloride, and 45S5 bioactive glass particles (BG) were added to the polymer solution (0, 25, and 50 weight % BG). The mixture was then poured into a 1% polyvinyl alcohol solution (sigma Chemicals, St. Louis) to form the microspheres. The microspheres were then washed, dried, and sifted into desired size ranges. The 3-D scaffold construct (7.5×18.5 mm) was formed by sintering the microspheres (300-350 μm) at 70° C. for over 6 hours.

Bovine articular chondrocytes were harvested aseptically from the carpometacarpal joints of 3 to 4-month old calves by enzymatic digestion [3]. The chondrocytes were plated and grown in fully supplemented Dulbecco's Modified Eagle Medium (DMEM, with 10% fetal bovine serum, 1% penicillin/streptomycin, 1% non-essential amino acids). The chondrocytes were maintained at 37° C., 5% CO₂ under humidified conditions.

The composites were sterilized by ethanol immersion and UV radiation. The scaffolds were seeded at 2.0×10⁵ cells/sample in 48-well plates. Samples (n=5) were maintained at 37° C. for 1, 7, 14, and 21 days. Cell proliferation, alkaline phosphatase (ALP) activity, glycosaminoglycan (GAG), and mineralization were examined in time.

The osteochondral construct consists of three regions, gel-only, gel/microsphere interface, and a microsphere-only region. Isolated bovine chondrocytes were suspended in 2% agarose (Sigma, MO.) at 60×10⁶ cells/ml. The PLAGA-BG scaffold was integrated with the chondrocyte-embedded agarose hydrogel using a custom mold. Chondrocytes were embedded in the gel-only region and osteoblasts were seeded on the microsphere-only region. All constructs were cultured in fully supplemented DMEM with 50 μg/ml of ascorbic acid. The cultures were maintained at 5% CO₂ and 37° C., and were examined at 2, 10, and 20 days.

Cell viability was assayed by a live/dead staining assay (Molecular Probe, OR.), where the samples were halved and imaged with a confocal microscope (Olympus, NY). Proliferation was measured using a fluorescence DNA assay, and ALP activity was determined by a calorimetric enzyme assay [2]. Cell morphology and gel-scaffold integration were examined at 15 kV using environmental scanning electron microscope (ESEM, FEI, OR.). For histology, samples were fixed in neutral formalin, embedded in PMMA and sectioned with a microtome. All sections were stained with hematoxylin and eosin, Picrosirius red for collagen, Alizarin Red S for mineralization, and Alcian Blue for GAG deposition.

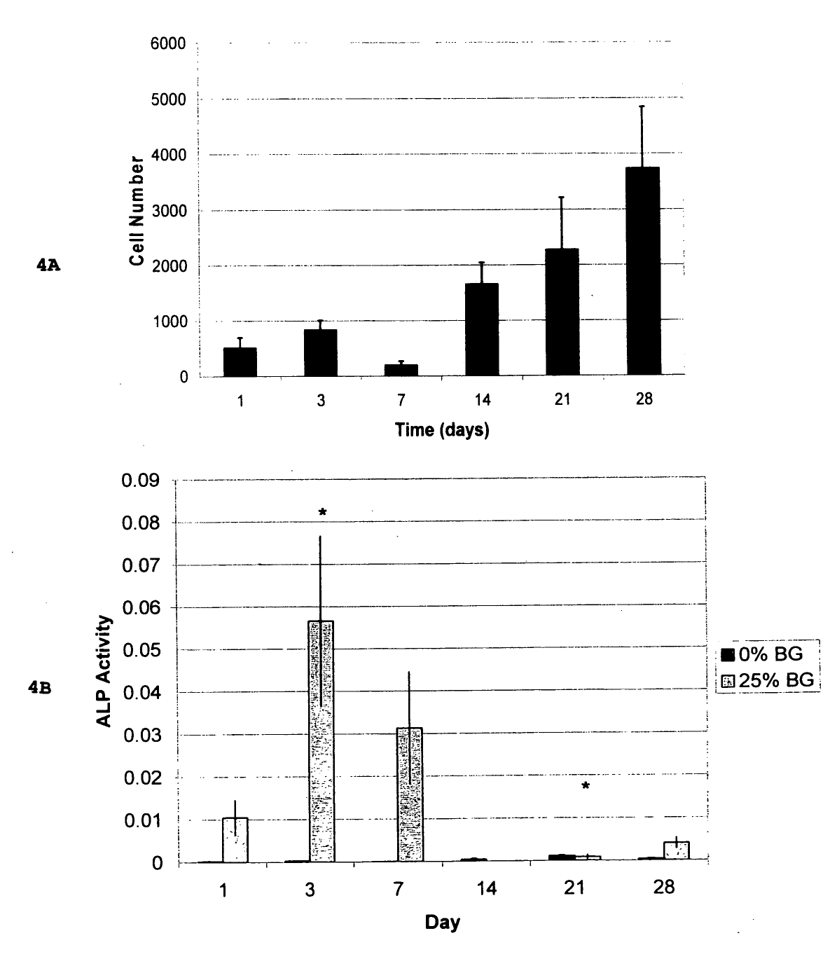

Chondrocytes maintained viability and proliferated on all substrates tested during the culture period (FIG. 4A). As shown in FIG. 4B, ALP activity of chondrocytes increased when grown on PLAGA-BG scaffolds, while a basal level of activity was observed on scaffolds without BG. Chondrocyte ALP activity peaked between days 3 and 7, and these cells elaborated a GAG-rich matrix on the PLAGA-BG composite scaffolds.

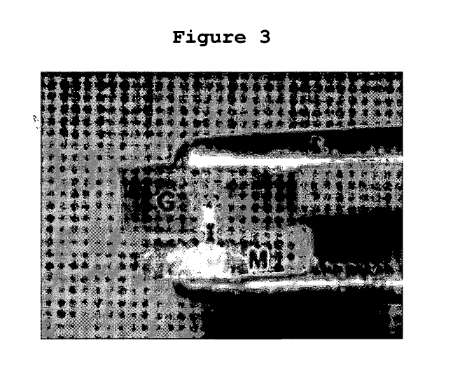

The agarose gel layer penetrated into the pores of the PLAGA-BG scaffolds and construct integrity was maintained over time, as seen in FIG. 3. Chondrocytes and osteoblasts remained viable in both halves of the construct for the duration of the culturing period.

Chondrocytes remained spherical in both the agarose-only region (G) and the interface (I) region. Chondrocytes (Ch) migrated out of the agarose hydrogel and they attached onto the microspheres in the interface region. These observations were confirmed as these migrating cells did not stain positively for the cell tracking dye used for the osteoblasts. Interestingly, chondrocyte migration was limited to the interface and no chondrocytes were observed in the microsphere region.

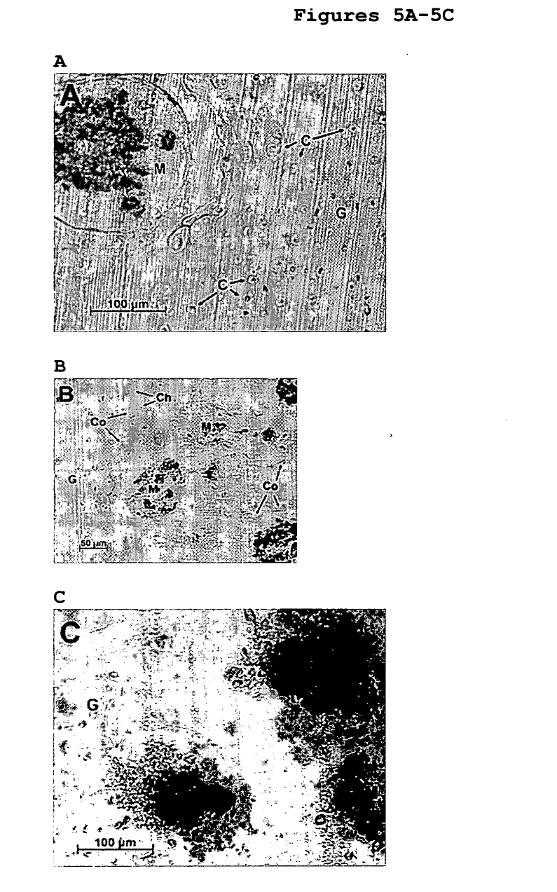

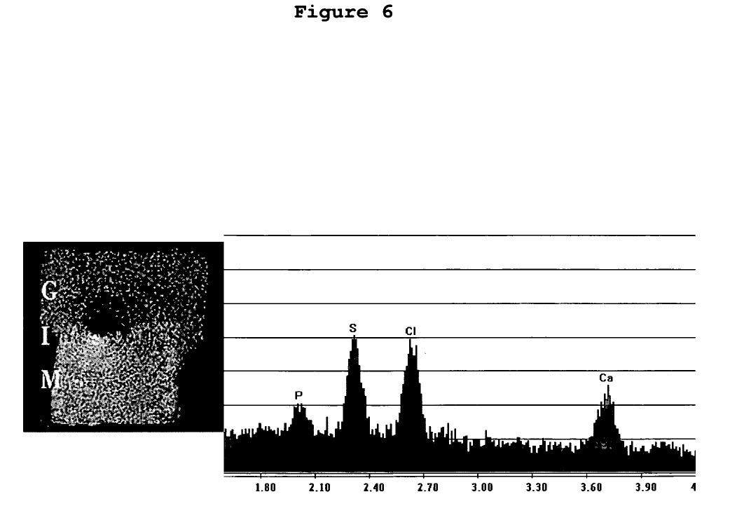

Collagen production was evident in both the gel (G) and microsphere (M) regions (FIG. 5B). As shown in FIG. 5A, positive Alcian Blue staining was observed at the interface (I) and within the gel (G), indicative of the deposition of a GAG-rich matrix within these regions by chondrocytes. A mineralized matrix was found within the microsphere region as well as the interface (FIGS. 5C, 6 left, 6 right). Energy dispersive x-ray analysis (EDAX) and microcomputerized tomography (micro-CT) scans revealed that the interfacial region is comprised of a mixture of GAG and amorphous calcium phosphate (FIG. 6).

This set of experiments focused on the development of a novel osteochondral graft for cartilage repair. Specifically, the PLAGA-BG composite and hydrogel scaffold consisted of a gel-only region for chondrogenesis, a microsphere-only region for osteogenesis, and a combined region of gel and microspheres for the development of an osteochondral interface.

In Experiment 1, the potential of the microsphere composite phase to support chondrocyte growth and differentiation was examined, as they are co-cultured with osteoblasts on the osteochondral scaffold. Cell viability and proliferation were maintained on the scaffolds during culture. In addition, the chondrocytes produced a GAG-rich matrix, suggesting that their chondrogenic potential was maintained in the presence of Ca—P. It is interesting to note that the PLAGA-BG composite promoted the ALP activity of chondrocytes in culture. ALP is an important enzyme involved in cell-mediated mineralization, and its heightened activity during the first week of culture suggest that chondrocytes may participate in the production of a mineralized matrix at the interface.

The osteochondral graft in Experiment 2 supported the simultaneous growth of chondrocytes and osteoblasts, while maintaining an integrated and continuous structure over time. The agarose hydrogel phase of the graft promoted the formation of the GAG-rich matrix. Chondrocytes embedded in agarose have been shown to maintain their phenotype [3, 4] and develop a functional extracellular matrix in free-swelling culture [3]. More importantly, the osteochondral graft was capable of simultaneously supporting the growth of distinct matrix zones—a GAG-rich chondrocyte region, an interfacial matrix rich in GAG, collagen, and a mineralized collagen matrix produced by osteoblasts. The pre-designed regional difference in BG content across the hybrid scaffold coupled with osteoblast-chondrocyte interactions may have mediated the development of controlled heterogenity on these scaffolds. Previously, such distinct zonal differentiations have only been observed on osteochondral grafts formed in vivo [5, 6]. A reliable in vitro osteochondral model will permit in-depth evaluation of the cell-mediated and scaffold-related parameters governing the formation of multiple tissue zones on a tissue engineered scaffold. Chondrocyte migration into the interface region suggests that these cells may play an important role in the development of a functional interface.

Second Set of Experiments

This set of experiments characterizes the growth and maturation of chondrocytes on composite scaffolds (PLAGA-BG) with varying composition ratios of poly-lactide-co-glycolide (PLAGA) and 45S5 bioactive glass (BG).

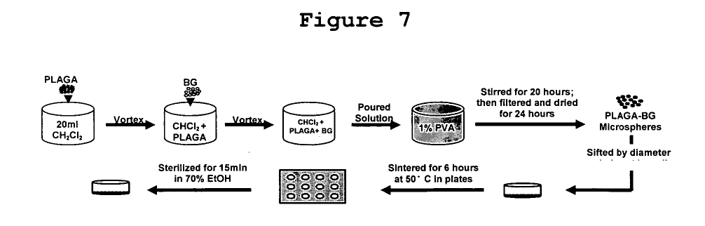

For the sample preparation, a water-oil-water emulsion was used (FIG. 7) [7].

Chondrocytes were harvested asceptically from the bovine carpametacarpal joints (˜1 week old). The cartilage was digested for 2 h with protease, 4 h with collagenase and resuspended in fully supplemented Dulbecco's Modified Eagle Medium (DMEM+10% serum+1% antibiotics+1% non-essential amino acids, 50 μg/ml ascorbic acid).

Composites seeded with cells (64,000 cells/samples) were maintained in a 37° C. incubator (5% CO₂).

At day 1, 3, 7, 14, 21 and 28 days, the samples were harvested and analyzed for cell proliferation (n=5), ALP activity (n=5), GAG deposition (n=5) and histology.

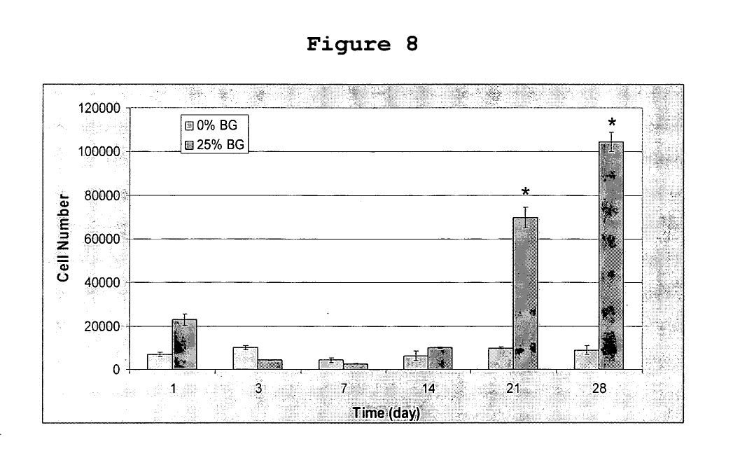

Chondrocytes were viable and proliferated on all substrates tested. A significantly higher number of cells attached to the 25% composite, and higher number of chondrocytes were found on the 25% samples after 28 days of culture (p<0.05) (FIG. 8).

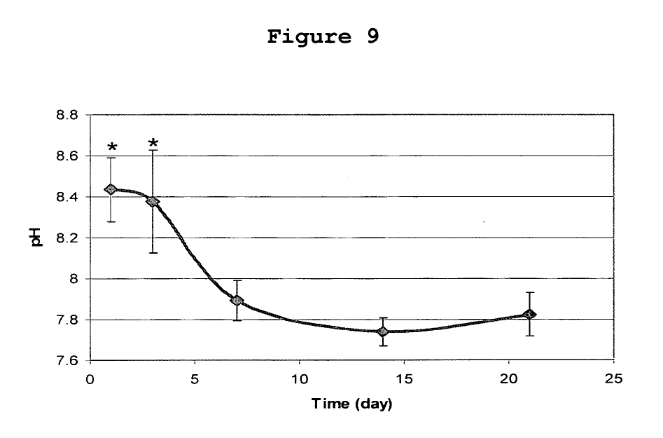

From days 1-7, cell number was lower on the 25% substrates (p>0.05), likely due to surface reactions occurring at the PLAGA-BG composite surface. Media pH measured significantly higher alkalinity at days 1 and 3 for 25% BG composites (p<0.05) (FIG. 9).

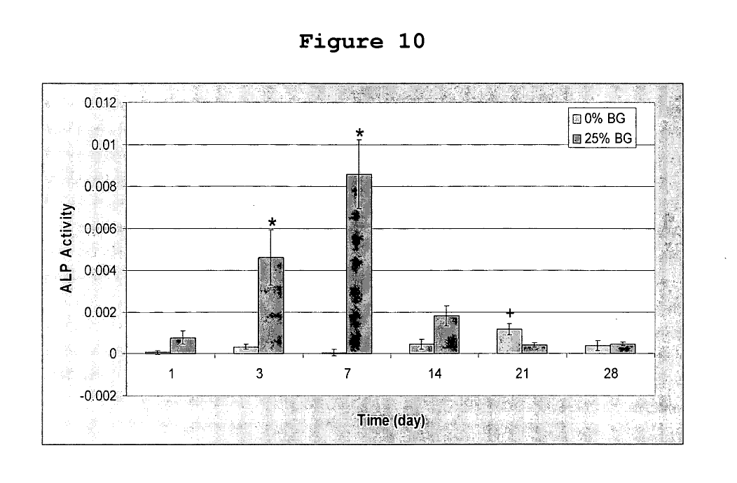

ALP activity was higher on the 25% PLAGA-BG samples (p<0.05) (FIG. 10). ALP activity peaked at day 7 for the 25% samples, as compared to day 21 for the 0% group (FIG. 10).

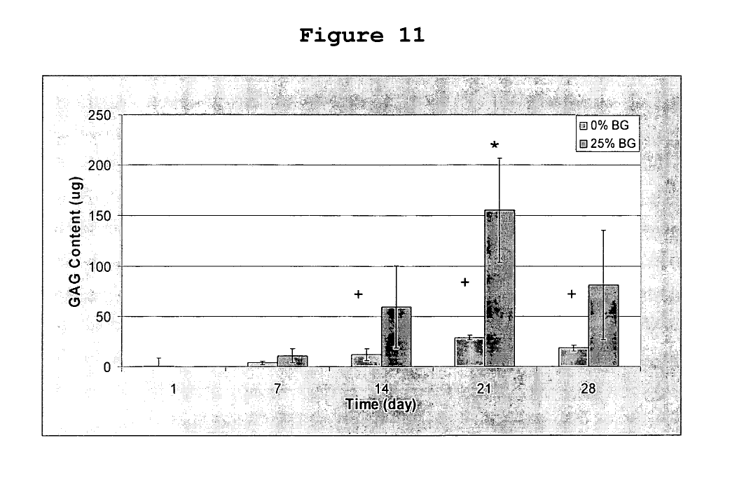

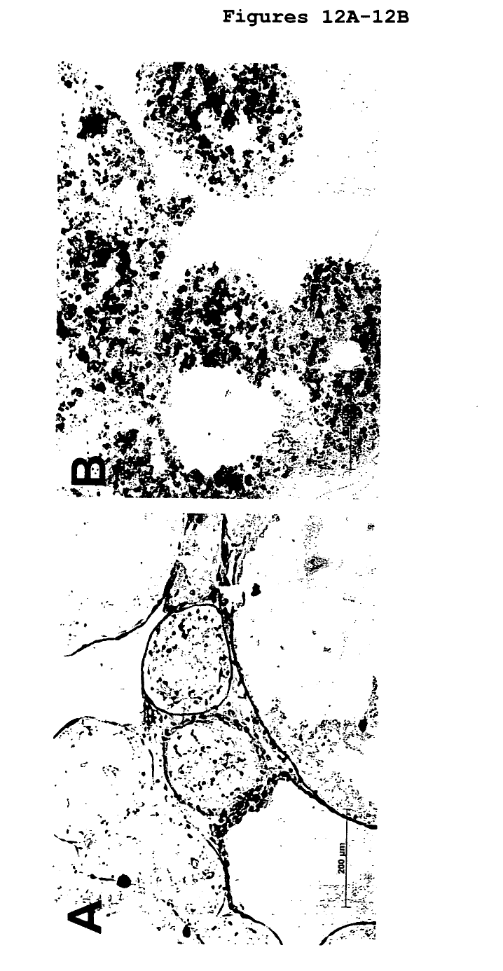

Chondrocytes continued to elaborate on GAG matrix, and GAG content increased with time and peaked on day 21 (FIG. 11). Chondrocytes penetrated and grew within the pores of the microsphere scaffolds. Mineralization nodules were found on chondrocytes grown on PLAGA-BG composites (FIG. 12).

The second set of experiments further show that PLAGA-BG composite supports chondrocyte proliferation and matrix deposition during the culturing period. The BG surface reactions which lead to the formation of a surface Ca—P layer [8] had a significant effect on the chondrocytes.

PLAGA-BG composites have been shown to be osteoconductive [8]. PLAGA-BG composite with 25% BG caused an increase in ALP activity in articular chondrocytes compared to the control which is consistent with the previous findings with 100% BG [9]. The BG induced mineralization seen here may mimic endochondral bone formation and may be used to facilitate the formation of tidemark in tissue engineered osteochondral grafts.

### REFERENCES

1. Hunziker, E. B., Osteoarthritis and Cartilage, 7:15-28 (1999).
2. Lu, H. H., et al., Journal of Biomedical Materials Research, 64A:465-474 (2003).
3. Mauck, R. L., et al., Osteoarthritis and Cartilage, 11:879-890 (2003).
4. Benya and Shaffer, “Dedifferentiated chondrocytes reexpress the differentiated collagen phenotype when cultured in agarose gels,” Cell. 30(1):215-24 (1982).
5. Gao, et al., “Repair of osteochondral defect with tissue-engineered two-phase composite material of injectable calcium phosphate and hyaluronan sponge,” Tissue Eng. 8(5):827-37 (2002).
6. Alhadlaq, A and Mao, J. J., Journal of Dental Research, 82:951-956 (2003).
7. Borden, et al., Biomat. 24:597-609 (2003).
8. Hench, et al., “Bioceramics: From concept to clinic,” J. Am. Ceram. Soc. 74(7): 1487-1510 (1991).
9. Asselin, et al., Biomat. 25:5621-5630 (2004).
10. Scapinelli, R. & Little, K., “Observations on the mechanically induced differentiation of cartilage from fibrous connective tissue,” J. Pathol. 101, 85-91 (1970).
11. Gregoire, M., Orly, I., Kerebel, L. M. & Kerebel, B., “In vitro effects of calcium phosphate biomaterials on fibroblastic cell behavior,” Biol. Cell 59, 255-260 (1987).
12. Cheung, H. S. & McCarty, D. J., “Mitogenesis induced by calcium-containing crystals. Role of intracellular dissolution,” Exp. Cell Res. 157, 63-70 (1985).
13. Wuthier, R. E., “Involvement of cellular metabolism of calcium and phosphate in calcification of avian growth plate cartilage,” J. Nutr. 123, 301-309 (1993).
14. Gao, J. & Messner, K., “Quantitative comparison of soft tissue-bone interface at chondral ligament insertions in the rabbit knee joint,” J. Anat. 188, 367-373 (1996).
15. Jiang, J., Nicoll, S. B. & Lu, H. H., “Effects of Osteoblast and Chondrocyte Co-Culture on Chondrogenic and Osteoblastic Phenotype In Vitro,” Trans. Orhtop. Res. Soc. 49 (Abstract) (2003).
16. Spalazzi, J. P., Dionisio, K. L., Jiang, J. & Lu, H. H., “Chondrocyte and Osteoblast Interaction on a Degradable Polymer Ceramic Scaffold,” ASME 2003 Summer Bioengineering Conference (Abstract) (2003).
17. Sherwood, et al., “A three-dimensional osteochondral composite scaffold for articular cartilage repair,” Biomaterials. 23(24):4739-51 (2002).
18. Schafer, et al., “In vitro generation of osteochondral composites,” Biomaterials 21:2599-2606 (2000).
19. Schafer, et al., “Tissue-engineered composites for the repair of large osteochondral defects,” Arthritis Rheum. 46:2524-2534 (2002).
20. Watt and Dudhia, “Prolonged expression of differentiated phenotype by chondrocytes cultured at low density on a composite substrate of collagen and agarose that restricts cell spreading,” Differentiation. 38(2):140-7 (1998).
21. Buschmann, et al., “Chondrocytes in agarose culture synthesize a mechanically functional extracellular matrix,” J Orthop Res. 10(6):745-58 (1992).
22. Buschmann, et al., “Mechanical compression modulates matrix biosynthesis in chondrocyte/agarose culture,” J Cell Sci. 108 (Pt 4):1497-508 (1995).
23. Lee and Bader, “The development and characterization of an in vitro system to study strain-induced cell deformation in isolated chondrocytes,” In Vitro Cell Dev Biol Anim. 31(11):828-35 (1995).
24. Chang and Pole, “Confocal analysis of the molecular heterogeneity in the pericellular microenvironment produced by adult canine chondrocytes cultured in agarose gel,” Histochem J. 29(7):515-28 (1997).
25. Rahfoth, et al., “Transplantation of allograft chondrocytes embedded in agarose gel into cartilage defects of rabbits,” Osteoarthritis Cartilage. 6(1):50-65 (1998).
26. Sittinger, et al., “Engineering of cartilage tissue using bieresorbable polymer carriers in perusion culture,” Biomaterials 15(6):451-456 (1994).
27. Borden, et al., “The sintered microsphere matrix for bone tissue engineering: in vitro osteoconductivity studies,” J Biomed Mater Res. 61(3):421-9 (2002).
28. Hench, et al., “An investigation of bioactive glass powders by sol-gel processing,” J Appl Biomater. 2(4):231-9 (1991).
29. Ducheyne, et al., “Effect of bioactive glass templates on osteoblast proliferation and in vitro synthesis of bone-like tissue,” J Cell Biochem. 56(2):162-7 (1994).

What is claimed is:

1. An apparatus for osteochondral tissue engineering, wherein said apparatus comprises regions of varying matrices which provide a functional interface between multiple tissue types, said regions comprising:
(a) a first region comprising a hydrogel;
(b) a second region adjoining the first region; and
(c) a third region adjoining the second region and comprising a porous scaffold.

2. The apparatus of claim 1, wherein the apparatus promotes growth and development of multiple tissue types.

3. The apparatus of claim 1, wherein the first region is seeded with cells for chondrogenesis, the third region is seeded with cells for osteogenesis, and the scaffold apparatus comprising the first region seeded with the cells for chondrogenesis, and the third region seeded with the cells for osteogenesis is maintained in an environment supporting migration of at least some of the cells for chondrogenesis into the second region and migration of at least some of the cells for osteogenesis into the second region.

4. The apparatus of claim 3, wherein the cells for chondrogenesis include chondrocytes.

5. The apparatus of claim 4, wherein the chondrocytes are selected from the group comprising surface zone chondrocytes, middle zone chondrocytes or deep zone chondrocytes.

6. The apparatus of claim 3, wherein the cells for chondrogenesis include stem cells.

7. The apparatus of claim 3, wherein the cells for osteogenesis include osteoblasts and/or osteoblast-like cells.

8. The apparatus of claim 3, wherein the cells for osteogenesis include stem cells.

9. The apparatus of claim 1, wherein the first region supports the growth and maintenance of cartilage tissue, the third region supports the growth and maintenance of bone tissue, and the second region functions as an osteochondral interfacial zone.

10. The apparatus of claim 3, wherein the first region is rich in glycosaminoglycan.

11. The apparatus of claim 1, one or more agents selected from a group comprising the following are introduced in said first region: anti-infectives; hormones, analgesics; anti-inflammatory agents; growth factors; chemotherapeutic agents; anti-rejection agents; and RGD peptides.

12. The apparatus of claim 11, wherein the growth factor is Transforming Growth Factor-beta (TGF-beta).

13. The apparatus of claim 1, wherein the hydrogel of the first region is agarose hydrogel.

14. The apparatus of claim 1, wherein the second region supports the growth and maintenance of fibrocartilage.

15. The apparatus of claim 1, wherein the second region includes a combination of hydrogel and the porous scaffold.

16. The apparatus of claim 14, wherein the second region is rich in glycosaminoglycan and collagen.

17. The apparatus of claim 1, wherein one or more growth factors selected from the following are introduced into the second region: Transforming Growth Factor-beta (TGF-beta), parathyroid hormone and insulin-derived growth factors (IGF).

18. The apparatus of claim 1, wherein the third region for supporting the growth and maintenance of bone tissue is seeded with at least one of osteoblasts, osteoblast-like cells and stem cells.

19. The apparatus of claim 1, wherein the third region includes a mineralized collagen matrix.

20. The apparatus of claim 1, wherein the third region contains at least one of osteogenic agents, osteogenic materials, osteoinductive agents, osteoinductive materials, osteoconductive agents, osteoconductive materials, growth factors and chemical factors.

21. The apparatus of claim 20, wherein the growth factors are selected from the group comprising Transforming Growth Factor-beta (TGF-beta), bone morphogenetic proteins, vascular endothelial growth factor, platelet-derived growth factor and insulin-derived growth factors (IGF).

22. The apparatus of claim 1, wherein the porous scaffold comprises a composite of polymer and ceramic.

23. The apparatus of claim 22, wherein the ceramic is bioactive glass.

24. The apparatus of claim 22, wherein the ceramic is calcium phosphatase.

25. The apparatus of claim 23, wherein the third region contains approximately 25% bioactive glass by weight.

26. The apparatus of claim 22, wherein a gradient of calcium phosphate concentrations appears across the first, second and third regions.

27. The apparatus of claim 26, wherein the gradient of calcium phosphate concentration is related to the percent of bioactive glass by weight in the third region

28. The apparatus of claim 26, wherein the calcium phosphate is selected from the group comprising tricalcium phosphate, hydroxyapatite and a combination thereof.

29. The apparatus of claim 22, wherein the polymer in the third region is selected from the group comprising aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, and biopolymers, and a blend of two or more of the preceding polymers.

30. The apparatus of claim 29, wherein the polymer comprises at least one of the poly(lactide-co-glycolide), poly(lactide) and poly(glycolide).

31. The apparatus of claim 1, wherein the apparatus is biodegradable.

32. The apparatus of claim 1, wherein the apparatus is osteointegrative.

33. A method for treating osteochondral tissue injury in a subject comprising grafting the apparatus of claim 1 with a co-culture of two or more cells selected from the group comprising chondrocytes, osteoblasts, osteoblast-like cells and stem cells in the subject at the location of osteochondral injury.

34. The method of claim 33, wherein the osteochondral tissue injury is craniofacial tissue injury.

35. The method of claim 33, wherein the osteochondral tissue injury is musculoskeletal tissue injury.

36. The method of claim 33, wherein the chondrocytes are selected from the group comprising surface zone chondrocytes, middle zone chondrocytes and deep zone chondrocytes.

37. A method for treating cartilage degeneration in a subject comprising grafting the apparatus of claim 1 with a co-culture of two or more cells selected from the group comprising chondrocytes, osteoblasts, osteoblast-like cells and stem cells in the subject at the location of cartilage degeneration.

38. The method of claim 37, wherein the cartilage degeneration is caused by osteoarthritis.

39. The method of claim 37, wherein the chondrocytes are selected from the group comprising surface zone chondrocytes, middle zone chondrocytes and deep zone chondrocytes.

40. A method for evaluating cell-mediated and scaffold-related parameters of development and maintenance of multiple tissue zones in vitro comprising:
(a) co-culturing cells of different tissue on the apparatus of claim 1;
(b) after a suitable period of time, examining the development and maintenance of the cells on the apparatus.

41. The method of claim 40, wherein the cells of different tissues comprise two or more of the cells selected from the group comprising chondrocytes, osteoblasts, osteoblast-like cells and stem cells.

42. The method of claim 41, wherein the chondrocytes are selected from the group comprising surface zone chondrocytes, middle zone chondrocytes and deep zone chondrocytes.

43. The method of claim 40, wherein the cell-mediated and scaffold related parameters of development and maintenance comprise cell proliferation, alkaline phosphatase activity, glycosaminoglycan deposition, mineralization, cell viability, scaffold integration, cell morphology, phenotypic expression, and collagen production.

44. A method for preparing an apparatus for osteochondral tissue engineering, said method comprising the steps of:
(a) using a mold to form an apparatus comprising a first region comprising hydrogel, a second region adjoining said first region, and a third region adjoining said second region and comprising a porous scaffold;
(b) seeding said first region with one or more cells for chondrogenesis;
(c) seeding said third region with one or more cells for osteogenesis; and
(d) maintaining the apparatus comprising the first region seeded with the cells for chondrogenesis and the third region seeded with the cells for osteogenesis in an environment supporting migration of at least some of the cells for chondrogenesis into the second region and migration of at least some of the cells for osteogenesis into the second region.

45. The method of claim 44, wherein said cells for chondrogenesis include chondrocytes.

46. The method of claim 45, wherein the chondrocytes are selected from the group comprising surface zone chondrocytes, middle zone chondrocytes and deep zone chondrocytes.

47. The method of claim 44, wherein said cells for chondrogenesis include stem cells.

48. The method of claim 44, wherein the first region supports the growth and maintenance of cartilage tissue, the third region supports the growth and maintenance of bone tissue, and the second region functions as an osteochondral interfacial zone.

49. The method of claim 44, wherein said cells for osteogenesis include osteoblasts and/or osteoblast-like cells.

50. The method of claim 44, wherein said cells for osteogenesis include stem cells.

51. The method of claim 44, wherein the first region is rich in glycosaminoglycan.

52. The method of claim 44, further comprising the step of introducing in said first region one or more agents selected from a group comprising the following: anti-infectives; hormones; analgesics; anti-inflammatory agents; growth factors; chemotherapeutic agents; anti-rejection agents; and RGD peptides.

53. The method of claim 52, wherein the growth factor is Transforming Growth Factor-beta (TGF-beta).

54. The method of claim 44, wherein the hydrogel of the first region is agarose hydrogel.

55. The method of claim 44, wherein the second region supports the growth and maintenance of fibrocartilage.

56. The method of claim 44, wherein the second region includes a combination of hydrogel and the porous scaffold

57. The method of claim 55, wherein the second region is rich in glycosaminoglycan and collagen.

58. The method of claim 44, wherein one or more growth factors selected from the following are introduced into the second region: Transforming Growth Factor-beta (TGF-beta), parathyroid hormone and insulin-derived growth factors (IGF).

59. The method of claim 44, wherein the third region includes a mineralized collagen matrix.

60. The method of claim 44, wherein the third region contains at least one of osteogenic agents, osteogenic materials, osteoinductive agents, osteoinductive materials, osteoconductive agents, osteoconductive materials, growth factors and chemical factors.

61. The method of claim 60, wherein the growth factors are selected from the group comprising Transforming Growth Factor-beta (TGF-beta), bone morphogenetic proteins, vascular endothelial growth factor, platelet-derived growth factor and insulin-derived growth factors (IGF).

62. The method of claim 44, wherein the porous scaffold comprises a composite of polymer and ceramic.

63. The method of claim 62, wherein the ceramic is bioactive glass.

64. The method of claim 62, wherein the ceramic is calcium phosphatase.

65. The method of claim 63, wherein the third region contains approximately 25% bioactive glass by weight.

66. The method of claim 62, wherein a gradient of calcium phosphate concentrations appear across said first, second and third regions.

67. The method of claim 66, wherein the gradient of calcium phosphate concentrations is related to the percent of bioactive glass by weight in the third region.

68. The method of claim 66, wherein the calcium phosphate is selected from the group comprising tricalcium phosphate, hydroxyapatite, and a combination thereof.

69. The method of claim 62, wherein the polymer in the third region is selected from the group comprising aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, and biopolymers, and a blend of two or more of the preceding polymers.

70. The method of claim 69, wherein the polymer comprises at least one of poly(lactide-co-glycolide), poly(lactide) and poly(glycolide).

71. The method of claim 44, wherein the apparatus prepared though said method is biodegradable.

72. The method of claim 44, wherein the apparatus prepared through said method is osteoinductive.
